(19) United States
(12) Patent Application Publication
(10) Pub. No.: US 2024/0382440 A1
(43) Pub. Date: Nov. 21, 2024

(54) COMPOSITIONS OF POLYLYSINE CONJUGATES AND OF MICELLES AND/OR COPOLYMERS OF POLYLYSINE

(71) Applicant: HYDRO FILL TECHNOLOGY, BOULIAC (FR)

(72) Inventor: Jean-Pascal ZAMBAUX, BOULIAC (FR)

(21) Appl. No.: 18/289,784

(22) PCT Filed: May 10, 2022
(86) PCT No.: PCT/EP2022/062634

§ 371 Date: Jul. 8, 2024

(30) Foreign Application Priority Data
May 10, 2021 (FR) FR 2104925

(51) Int. Cl. A61K 31/198; A61K 31/575; A61K 47/64; A61K 47/69

(52) U.S. Cl. CPC A61K 31/198; A61K 31/575; A61K 47/645; A61K 47/6907

(57) ABSTRACT

The present invention relates to a composition comprising:-at least one molecule covalently conjugated to a polylysine, and-at least one hydrophobic molecule which cannot be covalently conjugated to a polylysine, said molecule being encapsulated in a micelle, and/or at least one molecule conjugated to a polylysine by polymerization, said molecule and the polylysine forming a copolymer. The subject matter of the invention is also the method for producing said composition and/or the uses thereof.

### TECHNICAL FIELD

The invention relates to compositions of molecules, some being conjugated to specific polymers and others, which cannot be conjugated directly to these polymers, are in the form of copolymers or are encapsulated in micelles.

### PRIOR ART

In order to increase the efficacy and bioavailability of certain molecules in therapeutic, nutritional or cosmetic compositions, it is known practice to combine said molecules with specific polymers such as polylysine, in particular poly-L-lysine.

Polylysine is a homopolypeptide, which can be linear, and belongs to the group of cationic polymers. At neutral pH, polylysine contains a positively charged hydrophilic amino group. The precursor amino acid, lysine, contains two amino groups, one on the α-carbon and the other on the ε-carbon. During the development of the PNOS project, PTS produced iPr-PLys. HBr, in which the counterion for the terminal ammonium is a bromide ion.

Polylysine enhances the electrostatic interaction between the negatively charged ions of the cell membrane and the positively charged ions of the attachment factors at the surface of the culture. When adsorbed at the surface of the culture, it increases the number of positively charged sites available for the attachment of cells. Polylysine has a high positive charge density which allows it to form soluble complexes with negatively charged macromolecules (Park, Tae Gwan; Jeong, Ji Hoon; Kim, Sung Wan (2006 Jul. 7). “Current status of polymeric gene delivery systems”. Advanced Drug Delivery Reviews. 58 (4): 467-486. doi: 10.1016/j.addr.2006.03.007. ISSN 0169-409X. PMID 16781003). Homopolymers or block copolymers of polylysine have been widely used in the delivery of DNA (Kadlecova, Zuzana; Rajendra, Yashas; Matasci, Mattia; Baldi, Lucia; Hacker, David L.; Wurm, Florian M.; Klok, Harm-Anton (2013 Aug. 10). “DNA delivery with hyperbranched poly-lysine: a comparative study with linear and dendritic poly-lysine”. Journal of Controlled Release. 169 (3): 276-288. doi: 10.1016/j.jconrel.2013.01.019. ISSN 1873-4995. PMID 23379996) and proteins (Jiang, Yuhang; Arounleut, Phonepasong; Rheiner, Steven; Bae, Younsoo; Kabanov, Alexander V.; Milligan, Carol; Manickam, Devika S. (2016 Jun. 10). “SOD1 nanozyme with reduced toxicity and MPS accumulation”. Journal of Controlled Release. 231:38-49. doi: 10.1016/j.jconrel.2016.02.038. ISSN 1873-4995. PMID 26928528).

Polylysine, as a highly cationic macromolecule, is efficiently transported into cells by endocytosis (Hugues J-P. Ryser, Iain Drimmond, Wei-ChiangShen. The cellular uptake of horseradish peroxidase and its poly (lysine) conjugate by cultured fibroblasts is qualitatively similar despite a 900-fold difference in rate, 1982, https://doi.org/10.1002/jcp.1041130126). This absorption is preceded by strong adsorption at the surface of the cells, which is due to a non-specific interaction of the positive charges of the polymer with the negative charges present at the surface of most mammalian cells. The membrane transport of polylysine can therefore be described as non-specific absorptive endocytosis, as opposed to fluid endocytosis or receptor-mediated endocytosis.

Given its polypeptide nature, polylysine should biodegrade physiologically under the hydrolytic action of several proteolytic enzymes, such as the endopeptidase trypsin, chimotrypsin or aminopeptidase endopeptidases (Hudecz F, Kutassi-Kovács S, Mezö G, Szekerke M. Biodegradability of synthetic branched polypeptide with poly(L-lysine) back-bone. Biol Chem Hoppe Seyler. 1989;370 (9): 1019-1026. doi: 10.1515/bchm3.1989.370.2.1019; Quong D, Yeo J N, Neufeld R J. Stability of chitosan and poly-L-lysine membranes coating DNA-alginate beads when exposed to hydrolytic enzymes. J Microencapsul. 1999;16 (1): 73-82. doi: 10.1080/026520499289329; The Action of Trypsin on Poly-lysine BY S. G. WALEY Aim J. WAT-SON, 1953.

Advantageously, polylysine increases the half-life of the molecules with which it is associated. In addition, due to its amphiphilic character, it allows the blood-brain barrier to be crossed.

A molecule can be conjugated to polylysine if suitable functional groups are present in the structure of said molecule such that conjugation to polylysine can be achieved orthogonally. In particular, the molecules should comprise functional groups suitable for conjugation to amino group residues within poly-L-lysine (the primary amine available in polylysines) such as an alcohol group, carboxylic acid group, aldehyde group, amine group, etc., and they should not comprise functional groups that are incompatible for conjugation to polylysine.

However, some molecules do not include functional groups that are suitable for orthogonal conjugation to the polylysine backbone and/or comprise functional groups that are incompatible with covalent conjugation to polylysine. This is the case in particular for certain highly hydrophobic molecules such as cholesterol, and it is also the case for amino acids. These molecules cannot be conjugated to polylysine and, when they are incorporated into compositions with polylysine, said molecules remain free and are not transported by the polylysine, resulting in their bioavailability being low and the same as that when they are administered alone.

### SUMMARY OF THE INVENTION

To address this issue, the invention proposes:
encapsulating the hydrophobic molecules which cannot be conjugated to polylysine in micelles, in particular encapsulating them, or trapping them, in micellar structures which are preferentially formed by polylysine conjugates, and
incorporating the other molecules which cannot be covalently conjugated, such as amino acids, into the polylysine backbone via copolymerization of N-carboxyanhydrides (NCA) monomers.

Thus, the invention relates to a liquid composition comprising:
at least one molecule (i) covalently conjugated to a polylysine, and
at least one hydrophobic molecule (ii) which cannot be covalently conjugated to a polylysine, said molecule (ii) being encapsulated in a micelle, and/or at least one molecule (iii) conjugated to a polylysine by polymerization, said molecule (iii) and the polylysine forming a copolymer.

Advantageously, the invention thus makes it possible to benefit from the advantages of polylysine for all molecules, including those which cannot be chemically conjugated to polylysine by methods known to a person skilled in the art. Thus, the efficacy, solubility and bioavailability of all molecules is improved.

In particular, this configuration makes it possible:
to increase the half-life of the active molecules of the composition in the body,
to target tissues or cells on which the molecules of the composition are to act,
to allow the active molecules of the composition to cross the blood-brain barrier and to enter cells, in particular neurons and motor neurons,
to increase the stability and bioavailability of the active ingredient.

The efficacy of the composition is therefore enhanced and it is possible to administer lower doses and reduce the acute or chronic toxicity of the active molecules contained in the composition.

The invention also relates to a method for producing compositions according to the invention.

The compositions according to the invention can be used as a drug alone or in combination with the use of at least one other composition, and the invention also relates to the compositions for their use as a drug.

Finally, the compositions according to the invention can be used as a food supplement or cosmetic composition, alone or in combination with the use of another composition, and the invention also relates to the use of these compositions as a food supplement or cosmetic composition.

Other features and advantages will become apparent from the detailed description of the invention, the examples and the figures that follow.

### BRIEF DESCRIPTION OF THE FIGURES

FIG. 1a shows a polymerization scheme: L-Met NCA and L-Lys NCA (trifluoroacetate) are mixed and polymerized to obtain the copolymer poly-(L-lysine-co-methionine). (DMF=dimethylformamide).

FIG. 1b shows a deprotection scheme: the copolymer of FIG. 1a is deprotected to obtain a poly-(L-lysine-co-methionine) in the form of a bromide salt.

FIG. 2a shows a polymerization scheme: L-Cys NCA (trityl) and L-Lys NCA (trifluoroacetate) are mixed and polymerized to obtain the copolymer poly-(L-lysine-co-L-cysteine).

FIG. 2b shows a deprotection scheme: the copolymer of FIG. 1a is deprotected to obtain a poly-(L-lysine-co-L-cysteine) in the form of a bromide salt. (DMF=dimethylformamide).

FIG. 3 shows a diagram of micelle formation according to one embodiment of the invention: 10=micelles; 12=polylysine; 14=covalently conjugated molecules (in particular fatty acids); 16=highly hydrophobic molecules which cannot be conjugated to polylysine (for example cholesterol, farnesyl-cysteine, coenzyme Q10, curcumin).

### DETAILED DESCRIPTION OF THE INVENTION

### Definitions

For the purposes of the invention, the term “animal” means any animal apart from humans.

For the purposes of the invention, the term “amphiphilic conjugate” means a conjugate formed by a hydrophobic molecule X and a hydrophilic polymer Y, in particular a polylysine, the conjugate thus having an amphiphilic character.

For the purposes of the invention, the term “molecule X conjugated to a polylysine” means a molecule X covalently bonded to a polylysine, this bond preferentially being an amide, urea or carbamate bond depending on the chemical nature of the molecule X.

### Compositions According to the Invention

The present invention therefore relates to a liquid composition comprising:
at least one molecule (i) covalently conjugated to a polylysine, and
at least one hydrophobic molecule (ii) which cannot be covalently conjugated to a polylysine, said molecule (ii) being encapsulated in a micelle, and/or at least one molecule (iii) conjugated to a polylysine by polymerization, said molecule (iii) and the polylysine forming a copolymer.

The composition according to the invention can thus comprise:
at least one molecule (i) covalently conjugated to a polylysine, and at least one hydrophobic molecule (ii) which cannot be covalently conjugated to a polylysine, said molecule (ii) being encapsulated in a micelle, or
at least one molecule (i) covalently conjugated to a polylysine, and at least one molecule (iii) conjugated to a polylysine by polymerization, said molecule (iii) and the polylysine forming a copolymer, or
at least one molecule (i) covalently conjugated to a polylysine, and at least one hydrophobic molecule (ii) which cannot be covalently conjugated to a polylysine, said molecule (ii) being encapsulated in a micelle, and/or at least one molecule (iii) conjugated to a polylysine by polymerization, said molecule (iii) and the polylysine forming a copolymer.

For all of the active molecules present in the compositions according to the invention, they may be molecules per se (example: ascorbic acid) and/or be a salt of these molecules (example: ascorbate) and/or be an ester of these molecules (example: ascorbic acid ester) and/or be an anhydride (example: ascorbic acid anhydride).

The composition according to the invention therefore comprises one or more molecules covalently conjugated to polylysine. This type of conjugate is known to a person skilled in the art, the bonds between a molecule and the polylysine being in particular amide, urea or carbamate bonds depending on the nature and the chemical structure of the molecule in question. Exemplary embodiments of amide, urea and carbamate bonds are described, for example, in:
Ryser H J, Shen W C. Conjugation of methotrexate to poly (L-lysine) as a potential way to overcome drug resistance. Cancer. 1980 Mar. 15;45(5 Suppl):1207-11 (amide bonds),
Zhuxian Z, Jianbin T, Qihang S, William J. A multifunctional PEG-PLL drug conjugate forming redox-responsive nanoparticles for intracellular drug delivery, Issue 38, 2015. Journal of Materials Chemistry B (amide bonds),
Scheper V, Wolf M, Scholl M, Kadlecova Z, Perrier T, Klok H A, Saulnier P, Lenarz T, Stöver T. Potential novel drug carriers for inner ear treatment: hyperbranched poly-lysine and lipid nanocapsules. Nanomedicine (Lond). 2009 August;4 (6): 623-35 (urea bonds),
Stéphanie Gac-Breton, Jean Coudane, Mahfoud Boustta & Michel Vert (2004) Norfloxacin-Poly(l-Lysine Citramide Imide) Conjugates and Structure-dependence of the Drug Release, Journal of Drug Targeting, 12:5, 297-307, (carbamate bonds),
Elmore, W. M. (2013). Nanoparticles Stabilized with MPEG-Poly-lysine Carbamate: Synthesis and Characterization, (carbamate bonds),
Ning-Ping Huang, Janos Vörös, Susan M. De Paul, Marcus Textor, and Nicholas D. Spencer. Biotin-Derivatized Poly(l-lysine)-g-poly(ethylene glycol): A Novel Polymeric Interface for Bioaffinity Sensing. Langmuir 2002 18 (1), 220-230 (carbamate bonds).

Preferentially, the one or more molecules covalently conjugated to a polylysine are selected from among fatty acids, antioxidants, vitamins and mixtures thereof. They can be selected in particular from among myristic acid, linoleic acid, palmitic acid, palmitoleic acid, lauric acid, oleic acid, orotic acid, spermine, taurine, pantothenic acid, azelaic acid, GABA, biotin, thioctic acid, ascorbic acid, acetic acid, propionic acid, butyric acid, pyruvic acid, lactic acid, alpha-tocopherol, pantothenic acid, glutathione, retinoic acid and mixtures thereof.

The composition according to the invention, in addition to these polylysine conjugates, can comprise hydrophobic molecules which cannot be conjugated to polylysine encapsulated in micelles. The hydrophobic molecules which cannot be conjugated to polylysine are chemically not suitable for covalent conjugation, either due to the absence of reactive functional groups or due to chemical incompatibilities. These molecules are highly hydrophobic; according to the invention, they are preferentially molecules having LogP values greater than 1, in particular greater than 2 and preferentially greater than 3. For example, they can be cholesterol, farnesyl-cysteine, coenzyme Q10 or curcumin. Preferentially, the one or more hydrophobic molecules which cannot be covalently conjugated to a polylysine are selected from among cholesterol, coenzyme Q10, farnesyl cysteine, curcumin, and mixtures thereof.

According to one preferred and particularly suitable embodiment, as shown in FIG. 3, the one or more hydrophobic molecules which cannot be covalently conjugated to a polylysine are encapsulated in at least one micelle 10 formed by amphiphilic conjugates each consisting of at least one hydrophobic molecule 14 covalently conjugated to a polylysine 12, encapsulating the one or more hydrophobic molecules 16 which cannot be covalently conjugated to a polylysine.

Specifically, given both the hydrophilic character of polylysine and the hydrophobic character of the majority of certain molecules suitable for covalent conjugation (in particular alpha-tocopherol or retinoic acid, or fatty acids such as oleic acid, palmitic acid, lauric acid, linoleic acid, azelaic acid, palmitoleic acid, thioctic acid, myristic acid, orotic acid, acetic acid, butyric acid, lactic acid, or propionic acid), then, according to the invention, these particular conjugates behave as surfactants in aqueous solution. In this way, some of these (amphiphilic) conjugates act as polymeric micelles in an aqueous medium, creating a hydrophobic pocket capable of encapsulating hydrophobic molecules which cannot be conjugated to polylysine (such as cholesterol, farnesyl cysteine, etc.), thereby considerably improving their solubility and stability in aqueous solution.

According to one particular embodiment of the invention, the one or more micelles encapsulating the hydrophobic molecules which cannot be conjugated to polylysine formed at least by one or more alpha-tocopherol conjugates covalently conjugated to a polylysine and/or by one or more retinoic acid conjugates covalently conjugated to a polylysine.

According to another particular embodiment of the invention, the one or more micelles encapsulating the hydrophobic molecules which cannot be conjugated to polylysine are formed at least by one or more fatty acid conjugates covalently conjugated to a polylysine. The one or more fatty acids are preferentially selected from among oleic acid, palmitic acid, lauric acid, linoleic acid, azelaic acid, palmitoleic acid, thioctic acid, myristic acid, orotic acid, acetic acid, butyric acid, lactic acid, and propionic acid.

The composition according to the invention, in addition to these polylysine conjugates, and optionally in addition to the hydrophobic molecules which cannot be conjugated to polylysine encapsulated in micelles, can comprise at least one molecule conjugated to a polylysine by polymerization, i.e. copolymers of said molecule and of polylysine. In particular, this molecule is selected from among amino acids such as cysteine or methionine for example. Specifically, amino acids, whether natural or not, cannot be conjugated to polylysine by covalent bonding. According to the invention, these molecules can be incorporated into the poly-L-lysine backbone via suitable copolymerization of NCA (N-carboxyanhydride) monomers to form a copolymer. Preferentially, molecules which cannot be conjugated to polylysine, in particular amino acids, are included in the polypeptide backbone of the polymer to form a copolymer.

Preferentially, the copolymers comprise between 5 and 20% molecules which cannot be covalently conjugated to polylysine (in particular amino acids) and between 80 and 95% polylysine. According to one preferred variant, the copolymers comprise 10% molecules which cannot be covalently conjugated to polylysine (in particular amino acids) and 90% polylysine (ratio of 1 to 10).

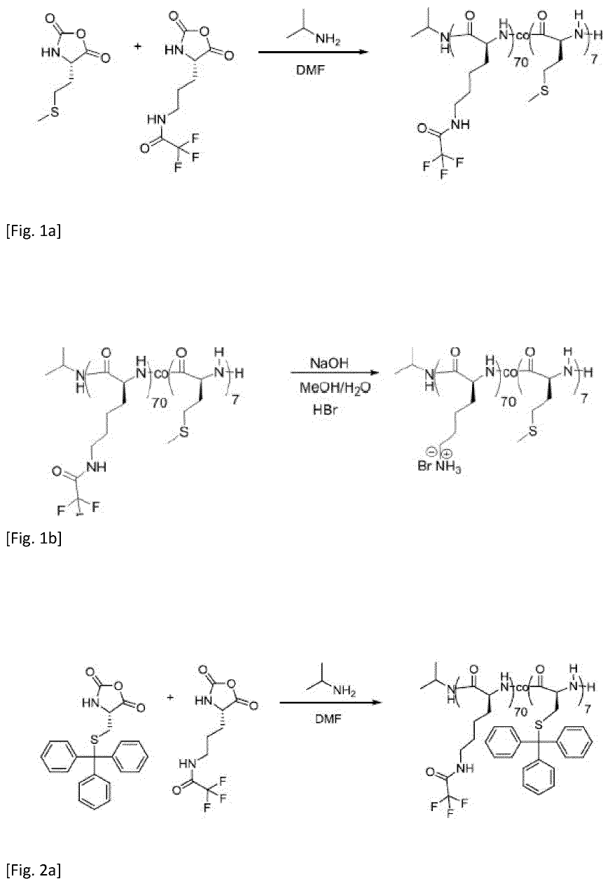

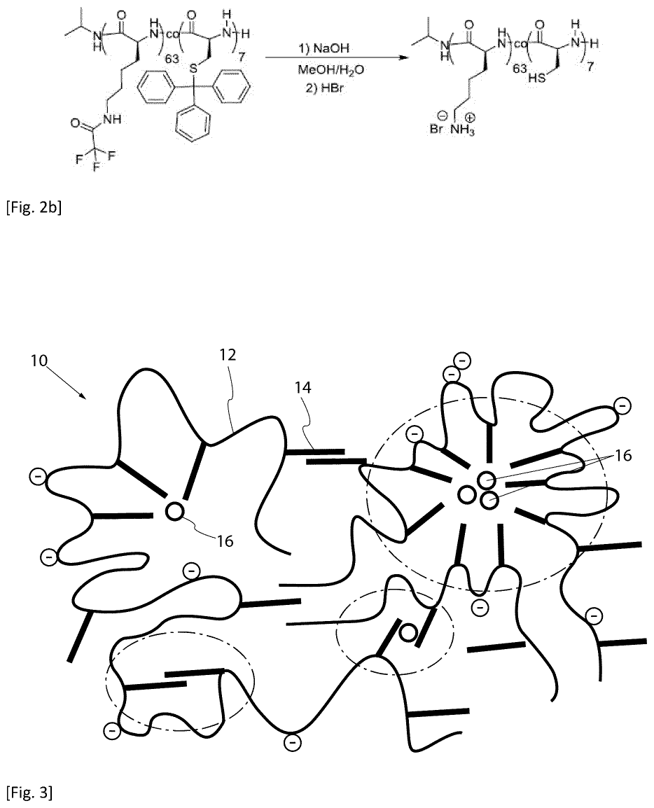

The copolymerization can be carried out in particular by the implementing a step of polymerization (for example L-Met NCA or L-Cys NCA and L-Lys NCA are mixed and polymerized to obtain a copolymer) followed by a step of deprotecting the copolymer obtained after polymerization. Illustrative examples are presented in FIGS. 1a, 1b (poly-(L-lysine-co-methionine), 2a and 2b (poly-(L-lysine-co-cysteine):
1a: Polymerization: The L-Met NCA and L-Lys NCA (trifluoroacetate) are mixed and polymerized in the appropriate ratio to obtain the appropriate random copolymer,
1b: Deprotection of PLys-TFA: The preceding copolymer is deprotected to obtain PLys in the form of a bromide salt.
2a: Polymerization: The L-Cys NCA (trityl) and L-Lys NCA (trifluoroacetate) are mixed in the appropriate ratio to obtain the appropriate random copolymer.
2b: Deprotection of Lys-TFA: The preceding copolymer is deprotected to obtain L-Lys in the form of a bromide salt and L-Cys in its deprotected form.

The polylysine used in the compositions according to the invention is preferentially a poly-L-lysine. The polylysine is preferentially linear. In particular, the polylysine used can be an epsilon poly-L-lysine, preferentially a poly-L-lysine with a molecular weight of between 12,000 and 20,000 Da. The polylysine used in the compositions according to the invention can be a polylysine that has bromide, chloride or TFA, trifluoroacetic acid, counterion.

The compositions according to the invention can be in solid form or in liquid form.

When in liquid form, the composition comprises at least water and the constituents mentioned above.

The solid form is preferentially obtained from the liquid form, preferentially by freeze-drying. Thus, when the composition according to the invention in liquid form comprises micelles and it is freeze-dried into solid form, the micelles reform when the composition in solid form is placed back into an aqueous solution.

Thus, the invention also relates to a solid composition, obtained by slow freeze-drying, spray-drying or dehydration of a liquid composition according to the invention.

The composition according to the invention can also comprise pharmaceutically or cosmetically acceptable excipients. These excipients can be selected in particular to meet the pH and osmolarity requirements of solutions for injection into humans or animals. For example, they can be acids or bases for adjusting pH or solutions for adjusting osmolarity, such as NaCl or PBS, phosphate-buffered saline.

The composition according to the invention is intended in particular to be administered to human beings, or to an animal, and is consequently in a form suitable for such administration. When it is in liquid form, it is preferentially suitable for subcutaneous or intravenous administration, in particular intravenous infusion, and it is packaged in suitable containers known to a person skilled in the art for packaging this type of product. The composition according to the invention can also be administered in liquid form via a pump, like an insulin pump.

When it is in solid form, it is preferentially suitable for administration:
transcutaneously and it is preferentially formulated and packaged in the form of a patch, or
nasally in the form of a powder, or
sublingually in the form of a powder or tablet, or
transmucosally, and it is preferentially formulated and packaged in the form of a mucoadhesive tablet.

One non-limiting exemplary composition according to the invention is a composition C1 comprising at least:
A. the following conjugates, each conjugate consisting of a molecule covalently bonded to a polylysine:
one or more oleyl-poly-L-lysine conjugates
one or more palmitic-poly-L-lysine conjugates
one or more lauryl-poly-L-lysine conjugates
one or more azelayl-poly-L-lysine conjugates
one or more palmitoleyl-poly-L-lysine conjugates
one or more thioctyl-poly-L-lysine conjugates
one or more myristyl-poly-L-lysine conjugates
one or more orotyl-poly-L-lysine conjugates
one or more acetate-poly-L-lysine conjugates
one or more buyrate-poly-L-lysine conjugates
one or more lactate-poly-L-lysine conjugates
one or more propionate-poly-L-lysine conjugates
one or more linoleyl-poly-L-lysine conjugates, and
B. farnesyl cysteine and cholesterol, and/or an ester of these molecules, encapsulated in micelles, preferentially in micelles formed by one or more of the conjugates listed under point A.

Preferentially:
the amount of butyric acid in composition C1 is greater than that of each of the other molecules taken individually, and/or
the amount of thioctic acid in composition C1 is greater than that of each of the other molecules taken individually, except for butyric acid, and/or
the amount of lauric acid in composition C1 is greater than that of each of the following molecules taken individually: palmitic acid, linoleic acid, azelaic acid, farnesyl cysteine, palmitoleic acid, cholesterol, myristic acid, and orotic acid,
the amount of oleic acid on the one hand, and of lactic acid on the other hand, in composition C1 is greater than that of each of the following molecules taken individually: palmitic acid, lauric acid, linoleic acid, azelaic acid, farnesyl cysteine, palmitoleic acid, cholesterol, myristic acid, orotic acid, and acetic acid.

Preferentially, each of the active molecules in composition C1 represents between 0.5 E−05 M and 10 E−05 M.

Another non-limiting exemplary composition according to the invention is composition C2 which comprises at least:
A. the following conjugates, each conjugate consisting of a molecule covalently bonded to a polylysine:
one or more taurine-poly-L-lysine conjugates
one or more spermine-poly-L-lysine conjugates
one or more pantothenyl-poly-L-lysine conjugates
one or more alpha-tocopherol-poly-L-lysine conjugates
one or more biotinyl-poly-L-lysine conjugates
one or more glutathione-poly-L-lysine conjugates
one or more ascorbyl-poly-L-lysine conjugates
one or more GABA-poly-L-lysine conjugates,
one or more retinoyl-poly-L-lysine conjugates, and
B. coenzyme Q10, and/or an ester and/or a salt of coenzyme Q10, encapsulated in micelles, preferentially in micelles formed by one or more of the conjugates listed under point A, and
C. methionine-poly-L-lysine copolymers and cysteine-poly-L-lysine copolymers, where the methionine and/or cysteine can be replaced with an ester, salt or anhydride of these molecules.

Preferentially:
the amount of retinoic acid in composition C2 is greater than that of each of the other molecules taken individually, and/or
the amount of taurine, the amount of methionine and the amount of GABA are equal in composition C2 and are each greater than that of each of the other molecules taken individually, except for retinoic acid and coenzyme Q10.

Preferentially, each of the active molecules in composition C2 represents between 6 E−05 M and 18 E−05 M.

### Method for Producing Compositions According to the Invention

The compositions according to the invention can be produced using any suitable method.

A particularly suitable production method comprises the following steps:
a. creating an amphiphilic premix: mixing one or more conjugates of a molecule which can be conjugated to polylysine in an aqueous solution (preferentially water),
b. adding at least one hydrophobic molecule which cannot be conjugated to polylysine (for example cholesterol, farnesyl-cysteine, coenzyme Q10, etc.) to the amphiphilic premix and stirring so as to form micelles formed by one or more of the conjugates of the amphiphilic premix encapsulating this or these one or more hydrophobic molecules which cannot be conjugated to polylysine, and/or
c. adding copolymers of polylysine and of molecules which cannot be covalently conjugated to polylysine, in particular amino acid-poly-L-lysine copolymers, to the mixture.

Thus, a method for producing a composition according to the invention comprises at least one hydrophobic molecule which cannot be covalently conjugated to a polylysine, encapsulated in a micelle formed by amphiphilic conjugates each consisting of at least one hydrophobic molecule covalently conjugated to a polylysine, characterized in that it comprises the following steps:
a. creating an amphiphilic premix: mixing conjugates of hydrophobic molecules covalently conjugated to a polylysine in an aqueous solution,
b. adding, to the amphiphilic premix, one or more hydrophobic one or more hydrophobic molecules which cannot be conjugated to a polylysine, and stirring so as to form a micelle of hydrophobic molecules covalently conjugated to a polylysine encapsulating the one or more hydrophobic molecules.

Specifically, one variant of the invention consists in taking advantage of the amphiphilic nature of certain polylysine conjugates (in particular fatty acid-polylysine conjugates), to create an amphiphilic premix that allows the controlled solubilization of highly hydrophobic molecules which cannot be conjugated to polylysine. The solubilization process according to one preferred embodiment consists in the controlled addition of the hydrophobic molecules to the amphiphilic premix and allowing the time needed for their dissolution with stirring.

Preferentially, the stirring in step b. is carried out for at least 5 minutes, even more preferentially between 5 and 20 minutes, and preferentially at a stirring speed of 900 revolutions per minute or less, in particular at a stirring speed of between 50 and 800 revolutions per minute.

Preferentially, the polylysine conjugates and other molecules are to be dissolved and formulated in water. It is also possible to add suitable salts such as PBS/NaCl afterwards, but these are not to be used before. Specifically, the use of salts such as PBS or NaCl in steps a. and/or b. prevents the formation of micelles and does not allow a clear solution to be obtained, and solubilization is not optimized. If PBS or NaCl is used, this should preferentially be after formation of the micelles and once the solution is clear.

According to one preferred embodiment, the production method according to the invention also comprises a step d of separating the soluble and insoluble phases, in order to recover the soluble phase. In this case, the insoluble phase is removed and the soluble phase constitutes the composition according to the invention. Specifically, a physical separation process (filtration, ultrafiltration) is preferentially carried out to ensure the isolation of the soluble fraction containing the amphiphilic premix, the copolymers and/or the dissolved encapsulated hydrophobic molecules.

According to one embodiment, the premix of step a. also comprises one or more copolymers of a molecule conjugated to a polylysine by polymerization and/or, in a step c. which is carried out after step b. or after step d., one or more copolymers of a molecule conjugated to a polylysine are added to the mixture.

Likewise, the premix of step a. can also comprise one or more other molecules covalently conjugated to a polylysine and/or, after step b. or d., one or more other molecules covalently conjugated to a polylysine are added to the mixture.

According to one variant, if the composition does not comprise micelles, the method for producing a composition according to the invention comprises at least one molecule conjugated to a polylysine by polymerization, said at least one molecule and the polylysine forming a copolymer, characterized in that it comprises mixing said at least one copolymer with at least one molecule covalently conjugated to a polylysine.

The liquid composition can then be freeze-dried or dehydrated to be in solid form. Specifically, the method according to the invention can comprise a step of slow freeze-drying, spray-drying or dehydration of the liquid composition obtained previously in order to obtain a solid composition, followed by a step of solubilization of this solid composition. Preferentially, the drying step is carried out by slow freeze-drying, for example between 12 and 36 hours.

Moreover, if polylysine conjugates and/or copolymers of the composition are not incorporated into the premix in step a or c, they can be added to the mixture after step b., i.e. after the formation of micelles and the encapsulation of coenzyme Q10.

The active molecule-polymer conjugates can be produced by any means known to a person skilled in the art for conjugating a molecule covalently to a polymer depending on their chemical nature. By way of example:
ascorbic acid is conjugated to polylysine via a carbamate bond,
spermine and taurine are conjugated to polylysine via a urea bond.
glutathione, pantothenic acid, aminobutyric acid, biotin, alpha-tocopherol, GABA, lauric acid, myristic acid, palmitic acid, oleic acid, linoleic acid, orotic acid, azelaic acid, thioctic acid, acetic acid, palmitoleic acid, butyric acid, lactic acid, propionic acid, retinoic acid and oleic acid are conjugated to polylysine via an amide bond.

The copolymers can be produced by any means known to a person skilled in the art. The molecules, in particular the amino acid molecules, are incorporated into the structure of the polymer preferentially in the presence of an anhydrous solvent and, according to one embodiment, in a ratio of 1 cysteine or methionine molecule to 10 monomers of the polymer.

### Uses

The compositions according to the invention can be used in particular as a drug, for preventing and/or treating diseases, in particular in humans or animals, i.e. for a human or animal subject, depending on the molecules they contain.

Likewise, the compositions according to the invention can be used as a food supplement or as a cosmetic composition.

Exemplary composition C1 is a generic formulation particularly suitable for preventing and/or treating multifactorial diseases, in particular multifactorial chronic diseases, and in particular diseases involving intestinal permeability.

It is capable of acting in a combined manner:
as an immunomodulator and autoimmune regulator, in particular by virtue of the presence of oleic acid, palmitic acid, palmitoleic acid,
as an anti-inflammatory, in particular by virtue of the presence of palmitic acid, azelaic acid, farnesyl cysteine, palmitoleic acid, myristic acid,
as an antibacterial, in particular by virtue of the presence of lauric acid, azelaic acid,
as an antifungal, in particular by virtue of the presence of lauric acid,
as an antiradical, in particular by virtue of the presence of farnesyl cysteine,
as an antagonist of isoprenylated proteins, in particular by virtue of the presence of farnesyl cysteine,
on the stabilization of cell membranes, in particular by virtue of the presence of cholesterol,
as a scavenger of free radicals and of metals, in particular by virtue of the presence of thioctic acid.
as an antiviral, in particular by virtue of the presence of myristic acid,
as a synergistic cell substrate, in particular by virtue of the presence of butyrate,
to promote the development of intestinal flora, in particular by virtue of the presence of orotic acid,
to regulate mucous membranes and intestinal flora, in particular by virtue of the presence of acetate, lactate, propionate and butyrate.

The various molecules present act synergistically and in particular make it possible to restore the function of intestinal permeability.

Thus, composition C1 can be used to restore the function of the intestinal membrane and/or to maintain intestinal permeability and/or to prevent or combat intestinal mucosa hyperpermeability.

According to one variant, composition C1 can be used as a food supplement in a healthy person or healthy animal, in particular to reduce the intestinal permeability of the person or animal to whom or which it is given.

The intestine plays a key role in the initiation and regulation of the chronicity of diseases. Thus, composition C1 can in particular be administered to humans or animals to prevent or combat diseases in which the intestine plays an important role, in particular chronic diseases, and in particular degenerative chronic diseases, such as neurodegenerative diseases like amyotrophic lateral sclerosis. Composition C1 can also be used as an immune system activator to prevent or combat autoimmune and infectious diseases.

The composition can be used in particular to reduce the chronicity of chronic diseases.

Preferentially, composition C1 is used in prevention or treatment as soon as the first symptoms of intestinal hyperpermeability or of a chronic disease appear, in order to prevent the development of the chronicity of said disease and thereby prevent the cytokine storm which results from prolonged intestinal hyperpermeability.

Specifically, composition C1 is particularly useful for at least:
reducing the inflammatory process, and/or
reducing the autoimmune process, and/or
reducing intestinal permeability, and/or
an antioxidant effect, and/or
an antiradical effect, and/or
an antibacterial and/or antiviral/or antifungal effect.

The invention therefore also relates to a composition C1 according to the invention for its use as a drug, in particular in humans or animals, i.e. for a human or animal subject.

In particular, composition C1 can be used in the prevention and/or treatment of a multifactorial disease, in particular a chronic multifactorial disease, in particular a disease selected from among inflammatory diseases, neurodegenerative diseases, bacterial diseases, viral diseases, autoimmune diseases, allergies and food intolerances. In particular, composition C1 can be used in the prevention and/or treatment of a disease selected from among eczema, psoriasis, chronic inflammatory intestinal diseases (in particular Crohn's disease and celiac disease), Alzheimer's disease, Parkinson's disease, multiple sclerosis, and amyotrophic lateral sclerosis.

Composition C1 is a formulation particularly suitable for preventing and/or treating multifactorial diseases, in particular multifactorial chronic diseases. Composition C2 is particularly useful for the prevention and treatment of neurodegenerative, autoimmune or infectious diseases, or cancers. In particular, it has great efficacy in the prevention and treatment of amyotrophic lateral sclerosis. Composition C2 intervenes in conventional and known processes to reduce the chronicity of diseases. Its action is particularly suited to neurodegenerative diseases, autoimmune diseases, cancer and infectious diseases.

In particular, it is capable of acting in a combined manner:
as an immunomodulator and autoimmune regulator, in particular by virtue of the presence of retinoic acid and GABA,
as a neuroprotector, in particular by virtue of the presence of taurine, coenzyme Q10, GABA
as an anti-inflammatory, in particular by virtue of the presence of spermine,
as cell protector, in particular by virtue of the presence of pantothenic acid, coenzyme Q10 (protection at the mitochondrial chain level in particular)
as a cell membrane protector, in particular by virtue of the presence of a-tocopherol,
as an antioxidant, in particular by virtue of the presence of taurine, cysteine, methionine, spermine, pantothenic acid, alpha-tocopherol, coenzyme Q10, glutathione, ascorbic acid,
as a cell metabolism regulator scavenging reactive oxygen species, in particular by virtue of ascorbic acid,
as a glutathione precursor, in particular by virtue of the presence of cysteine,
on the stabilization of the cell membranes,
as a scavenger of free radicals and metals, in particular by virtue of the presence of taurine, cysteine, methionine, spermine, pantothenic acid, coenzyme Q10, glutathione.

The various molecules present act synergistically and in particular make it possible to prevent or combat factors that are at the root of chronic diseases and amyotrophic lateral sclerosis in particular.

Thus, composition C2 can be used to prevent and/or combat chronic diseases, in particular to act on disease-specific metabolisms. Composition C2 can be used in particular to reduce the chronicity of chronic diseases.

Preferentially, composition C2 is used in prevention or treatment as soon as the first symptoms of a chronic disease, in particular a neurodegenerative disease and specifically amyotrophic lateral sclerosis, appear.

Composition C2 according to the invention can be used as a drug, in particular in humans or animals, i.e. for a human or animal subject.

In particular, composition C2 can be used in the prevention and/or treatment of a multifactorial disease, in particular a chronic multifactorial disease, in particular a disease selected from among inflammatory diseases, neurodegenerative diseases, bacterial diseases, viral diseases, autoimmune diseases, allergies and food intolerances, and in particular amyotrophic lateral sclerosis.

Composition C2 can be used together with composition C1, either simultaneously or with a time gap between them. In particular, it is preferable to administer composition C1 first, followed by composition C2, the two administrations preferentially being separated by at least 1 hour, even more preferentially by at least 4 hours, ideally one in the morning and one in the evening.

The invention is now described with the aid of examples and test results.

### EXAMPLES

### Example 1: Composition C1

An exemplary composition C1 suitable for testing on mice is presented below.

The following amounts of conjugates were weighed according to table 1 in order to prepare a stock of 300 ml of composition C1 10×. All of the conjugates were weighed using an analytical balance under a laminar flow hood.

TABLE 1

| PLL conjugate | |
|---|---|
| (PLL = poly-L-lysine) | Amount (mg) |
| Oleyl-PLL | 201 |
| Palmitic-PLL | 78 |
| Lauryl-PLL | 189 |
| Lynoleyl-PLL | 78 |
| Azelayl-PLL | 75 |
| Palmitoleyl-PLL | 156 |
| Thioctyl-PLL | 225 |
| Myristyl-PLL | 78 |
| Orotyl-PLL | 72 |
| Acetate-PLL | 174 |
| Buyrate-PLL | 285 |
| Lactate-PLL | 177 |
| Propionate-PLL | 177 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

In parallel, cholesterol and farnesyl cysteine molecules were dissolved separately at the approximate concentration of 50 mg/ml in absolute ethanol and filtered over a 0.22 μm filter. The solvent was evaporated using a rotary evaporator. The required amounts of dry solids were weighed under a laminar flow hood, as presented in table 2.

TABLE 2

| Molecule | Amount (mg) |
|---|---|
| Cholesterol | 12 |
| Farnesyl cysteine | 12 |

In a sterile 500 ml flask equipped with a magnetic bar, 23.8 ml of each conjugated solution prepared previously was added while being magnetically stirred (700 revolutions per minute). The corresponding amounts of (previously freeze-dried) salts at 300 of PBS were added to the solution of conjugates while being magnetically stirred (700 revolutions per minute).

Cholesterol and farnesyl cysteine were added and the mixture was stirred for 25 hours (700 revolutions per minute).

30 ml of formulation C1 10× was taken under a laminar flow hood and added to a new sterile 500 ml flask.

270 ml of sterile PBS was added to obtain formulation C1 1×. The formulation was stirred for 30 minutes.

The flasks were prepared according to the following plan using a sterile calibrated 5 ml pipette: C1 1×=83 flasks, 2 ml each.

C1 10×=83 flasks, 2 ml each.

The 166 flasks were placed in a steel freeze-drying unit and a freeze-drying cycle of one day was initiated.

### Example 2: Composition C2

An exemplary composition C2 suitable for testing on mice is presented below.

The following amounts of conjugates were weighed according to table 3 in order to prepare a stock of 300 ml of composition C2 10×. All of the conjugates were weighed using an analytical balance under a laminar flow hood.

TABLE 3

| PLL conjugate | |
|---|---|
| (PLL = poly-L-lysine) | Amount (mg) |
| Alpha-tocopherol-PLL | 210 |
| Ascorbyl PLL | 189 |
| Biotinyl-PLL | 204 |
| GABA-PLL | 378 |
| Glutathione-PLL | 222 |
| Pantothenyl-PLL | 204 |
| Retinoyl-PLL | 606 |
| Spermine-PLL | 198 |
| Taurine-PLL | 330 |
| PLL/Cysteine (copolymer) | 180 |
| PLL/Methionine (copolymer) | 303 |

Each conjugate was weighed in a sterile 50 ml centrifuge tube and was stored in the freezer at −20° C. until use.

In parallel, coenzyme Q10 was dissolved at the approximate concentration of 50 mg/ml in absolute ethanol and filtered through a 0.22 μm filter. The solvent was evaporated using a rotary evaporator. The required amounts of dry solids were weighed under a laminar flow hood, as presented in table 4.

TABLE 4

| Molecule | Amount (mg) |
|---|---|
| Coenzyme Q10 | 156 |

The solid was stored in a sterile centrifuge tube at −20° C. until use.

The 50 ml centrifuge tubes previously prepared with the conjugates and copolymers were taken and left to warm up to room temperature. After this time, 30 ml of water was added to each centrifuge tube.

The conjugates and copolymers were dissolved using a vortex mixer, stirring for a time of between 10 and 20 minutes depending on the solubility of the conjugates and copolymers.

The centrifuge tube containing coenzyme Q10 was also taken from the freezer and allowed to warm up to room temperature.

A sterile 500 ml flask was taken and provided with a magnetic bar. 27.3 ml of each conjugated solution prepared previously was added to the flask while being magnetically stirred (700 revolutions per minute).

The corresponding amounts of (previously freeze-dried) salts at 300 of PBS were added to the solution of conjugates while being magnetically stirred (700 revolutions per minute).

Coenzyme Q10 was added and the mixture was stirred for 2 hours (700 revolutions per minute).

After 2 hours, 30 ml of formulation C2 10× was taken under a laminar flow hood and added to a new sterile 500 ml flask.

270 ml of sterile PBS was added to obtain formulation C2×1. The formulation was stirred for 30 minutes.

The flasks were prepared according to the following plan using a sterile calibrated 5 ml pipette:

C2 1×=83 flasks, 2 ml each.

C2 10×=83 flasks, 2 ml each.

The 166 flasks were placed in the steel freeze-drying unit and a freeze-drying cycle of one day was initiated.

### Example 3: Composition C1

An exemplary composition C1 suitable for testing on rats is presented below.

The method for producing the composition is similar to that presented in example 1. The concentrations of each of the constituents are different and presented in table 5.

TABLE 5

| | Concentration | Concentration |
|---|---|---|
| Components | C1 10X (mg/mL) | C1 1X (mg/mL) |
| Acetate-PLL | 1.16 | 0.12 |
| Azelayl-PLL | 0.50 | 0.05 |
| Buyrate-PLL | 1.88 | 0.19 |
| Lauryl-PLL | 1.26 | 0.13 |
| Lactate-PLL | 1.18 | 0.12 |
| Linoleyl-PLL | 0.52 | 0.05 |
| Myristyl-PLL | 0.51 | 0.05 |
| Oleyl-PLL | 1.33 | 0.13 |
| Orotyl-PLL | 0.48 | 0.05 |
| Palmitic-PLL | 0.52 | 0.05 |
| Palmitoleyl-PLL | 1.04 | 0.10 |
| Propionate-PLL | 1.17 | 0.12 |
| Thioctyl-PLL | 1.49 | 0.15 |
| Cholesterol | 0.09 | 0.01 |
| FarCys | 0.07 | 0.01 |

The sum of the concentrations of the conjugates PLL+cholesterol+farnesyl cysteine for C1 10× is 13.2 mg/mL.

The sum of the concentrations of the conjugates PLL+cholesterol+farnesyl cysteine for C1 1× is 1.32 mg/ml.

### Example 4: Composition C1

An exemplary composition C1 suitable for humans is presented below.

The method for producing the composition is similar to that presented in example 1. The concentrations of each of the constituents are different and presented in table 6.

TABLE 6

| | Concentration | Concentration |
|---|---|---|
| Components | C1 10X (mg/mL) | C1 1X (mg/mL) |
| Acetate-PLL | 7.192 | 0.744 |
| Azelayl-PLL | 3.1 | 0.31 |
| Buyrate-PLL | 11.656 | 1.178 |
| Lauryl-PLL | 7.812 | 0.806 |
| Lactate-PLL | 7.316 | 0.744 |
| Linoleyl-PLL | 3.224 | 0.31 |
| Myristyl-PLL | 3.162 | 0.31 |
| Oleyl-PLL | 8.246 | 0.806 |
| Orotyl-PLL | 2.976 | 0.31 |
| Palmitic-PLL | 3.224 | 0.31 |
| Palmitoleyl-PLL | 6.448 | 0.62 |
| Propionate-PLL | 7.254 | 0.744 |
| Thioctyl-PLL | 9.238 | 0.93 |
| Cholesterol | 0.558 | 0.062 |
| FarCys | 7.192 | 0.744 |

### Example 5: Composition C2

An exemplary composition C2 suitable for humans is presented below.

The method for producing the composition is similar to that presented in example 2. The concentrations of each of the constituents are different and presented in table 7.

TABLE 7

| | Concentration | Concentration |
|---|---|---|
| Components | C1 10X (mg/mL) | C1 1X (mg/mL) |
| Alpha-tocopherol-PLL | 8.61 | 0.861 |
| Ascorbyl PLL | 7.749 | 0.7749 |
| Biotinyl-PLL | 8.364 | 0.8364 |
| GABA-PLL | 15.498 | 1.5498 |
| Glutathione-PLL | 9.102 | 0.9102 |
| Pantothenyl-PLL | 8.364 | 0.8364 |
| Retinoyl-PLL | 24.846 | 2.4846 |
| Spermine-PLL | 8.118 | 0.8118 |
| Taurine-PLL | 13.53 | 1.353 |
| PLL/Cysteine | 7.38 | 0.738 |
| (copolymer) | | |
| PLL/Methionine | 12.423 | 1.2423 |
| (copolymer) | | |
| Coenzyme Q10 | 6.396 | 0.6396 |

### Test Results

Study on the effect of composition C1 according to the invention on restoring intestinal permeability

The purpose of this study is to evaluate the efficacy of an injectable formulation according to the invention on the restoration of intestinal permeability following the induction of intestinal inflammation caused by dextran sulfate sodium (DSS) in rats.

The intestinal epithelium is a dynamic barrier that limits the access of bacteria, parasites, viruses and also certain chemical molecules to the tissues of the host. It is composed of different types of cells, each having a well-defined role, such as enterocytes which are responsible for nutrient absorption, goblet cells which secrete mucus, the thickness of which keeps bacteria and other microorganisms away from the enterocytes, Paneth cells which are located at the bottom of the crypts of the small intestine, and participate in maintaining homeostasis and in defending the intestinal mucosa via the secretion of antimicrobial agents such as defensins, and microfold cells, or M cells, which specialize in the endocytosis of antigens, molecules or microorganisms present in the intestinal lumen. They act as antigen-presenting cells since they present endocytized antigens to the underlying immune system. The epithelial physical barrier provided by the enterocytes is reinforced by a layer of glycocalyx and of thick mucus (this layer is thinner at M cells for easier access to the microbiome of the intestinal lumen) and secretory IgA via enterocyte transcytosis, produced by the underlying plasmocytes. The epithelial cells form a monolayer of polarized cells resting on the lamina propria via their basolateral poles and they are closely connected together by tight junctions. Peyer's patches are located in this lamina propria, these consisting of follicles rich in lymphocytes and are where pro-or anti-inflammatory responses are initiated. Many diseases are associated with a change in intestinal permeability, including chronic inflammatory bowel diseases, obesity, multiple sclerosis, amyotrophic lateral sclerosis and Alzheimer's disease. This change leads to an increase in intestinal permeability usually resulting in inflammation of the intestine wall.

The composition tested in this study is composition C1 from example 3.

The intestinal epithelium is a dynamic barrier that limits the access of bacteria, parasites, viruses and also certain chemical molecules to the tissues of the host. It is composed of different types of cells, each having a well-defined role, such as enterocytes which responsible for the absorption function of the intestine, goblet cells which secrete mucus, M cells which are responsible for transporting macromolecules and antigens from the intestinal lumen (lamina propria) to underlying immune cells and neuroendocrine cells. The epithelial physical barrier provided by the enterocytes is reinforced by a layer of glycocalyx and of thick mucus. The epithelial cells form a monolayer of polarized cells resting on the lamina propria via their basolateral poles and they are closely connected together by tight junctions. Many diseases are associated with a change in intestinal permeability, including chronic inflammatory bowel diseases, obesity, multiple sclerosis, amyotrophic lateral sclerosis and Alzheimer's disease. This change leads to an increase in intestinal permeability usually resulting in inflammation of the intestine wall.

The main objective of the study is to demonstrate the efficacy of the invention on the remediation of intestinal permeability following the induction of chronic intestinal inflammation.

To test these hypotheses, rats received different doses of composition C1 from example 3, administered by subcutaneous injection after induction of intestinal inflammation.

The procedure is described below.

### 1—Setting Up the Chronic Inflammation Model

After their arrival, the animals (Wistar rats, 280-300 g) were raised in 900 cm² cages (3 animals per cage). During this acclimatization period, the animals had free access to water and food. After this period, the animals (n=9) received, ad libitum, food and a solution of 2% (w/v) dextran sulfate sodium (DSS) in the drinking water. The induction of intestinal inflammation was monitored 3, 5 and 7 days after the start of the addition of DSS. A control group (n=3) received food and water ad libitum. If signs of pain were observed, the animal in question was isolated and paracetamol (200 mg/kg) was administered orally twice per day. These animals were removed from the experiment. If significant weight loss was observed, the animals were put down when they reached 80% of their initial weight. Lastly, animals were put down if they exhibited unusual behavior reflecting distress.

The conditions of accommodation were as follows: temperature 20-24° C., hygrometry 60 plus or minus 10%, 12 h/12 h cycle.

The animals were observed daily to watch for signs of stress or pain. Daily weighing of the animals, food and water was carried out. The appearance of clinical signs of inflammation was based on the loss of body weight, the consistency of stools (normal—0, soft—2, diarrhea—4) and rectal bleeding (0-4). The results made it possible to determine an inflammation severity index which represents the mean of the scores obtained for stool consistency and rectal bleeding. The weight loss represents the percentage difference in weight of the rats from D1 (addition of DSS) to D7.

### 2—Determining the “Effective” Dose

After their arrival, the animals (Wistar rats, 280-300 g) were raised in 900 cm² cages (5 animals per cage). During this acclimatization period, the animals had free access to water and food. After this period, the animals (n=30) were divided into 4 groups (Control−n=5, DSS+composition C1 10×−n=10). The “Control” group received food and water ad libitum throughout the experiment. The rats received, ad libitum, food and a solution of 2% DSS in the drinking water for 3, 5 or 7 days (duration determined during the first experiment). After the induction of intestinal inflammation, each rat received an intraperitoneal injection of 1 mL of composition C1. Composition C1 was administered once per day. If signs of pain were observed, the animal in question was isolated and paracetamol (200 mg/kg) was administered orally twice per day. These animals were removed from the experiment. If significant weight loss was observed, the animals were put down when they reached 80% of their initial weight. Lastly, animals were put down if they exhibited unusual behavior reflecting distress.

The conditions of accommodation were as follows: temperature 20-24° C., hygrometry 60 plus or minus 10%, 12 h/12 h cycle.

The animals were observed daily to watch for signs of stress or pain. Daily weighing of the animals, food and water was carried out.

The appearance of clinical signs of inflammation was based on the loss of body weight, the consistency of stools (normal—0, soft—2, diarrhea—4) and rectal bleeding (0-4).

The results made it possible to determine an inflammation severity index which represents the mean of the scores obtained for stool consistency and rectal bleeding. The weight loss represents the percentage difference in weight of the rats ((DX×D0)/D0)*100). At the end of the experiment, the animals were anesthetized (organ harvesting) and various samples were taken: blood, small intestine, cecum, colon, liver, spleen, and multiple parameters were evaluated: size of cecum, weight of liver, of spleen, and of cecum.

### Permeability Study

A permeability study was carried out on an isolated organ (jejunum or colon) in order to determine the efficacy of the dose of composition C1 on the recovery of intestinal permeability.

### Sampling Digestive Tract Fragments

This ex vivo approach used a fragment of jejunum or colon taken from the rat's digestive tract. The rats were anesthetized by inhaling isoflurane (1000 mg/g) at 3% for induction and 1.5% for maintenance during the operation. After laparotomy and ligation of the celiac artery against the esophagus, a 10 cm fragment of jejunum or colon was taken. The sample was weighed. The rats were then put down by injecting sodium pentobarbital (12 mg/100 g).

### Preparation of the Everted Organ and Sampling

The serosal and mucosal compartments of the organ were quickly rinsed with physiological saline solution (37° C.) and the organ was everted. A ligation was performed at one of the ends. The second end allows 1 mL of Krebs-Henseleit medium to be introduced. This end was then ligated, the organ was weighed and then placed in a container containing 10 mL of Krebs-Henseleit survival medium plus 250 mg of FITC-dextran, 4 kDa (mucosal compartment). Throughout the experiment, the everted organs were kept at 37° C. and oxygenated with an O2/CO2 mixture (95%/5%). Every 30 minutes, mucosal compartment medium was stirred by performing 3 suction-discharge cycles using a 1 mL micropipette. After 2 h of incubation, a sample of 1 mL was taken from the mucosal side.

The organs were removed from the glass container and one of the ends was cut. The serous medium was sampled and placed in a previously weighed tube. After sampling, the tube was again weighed in order to evaluate the transfer of survival medium over the course of the experiment. After weighing, the FITC-dextran was assayed by fluorimetry in duplicate (excitation, 490 nm; emission, 530 nm).

After weighing the organ, a longitudinal cut was made in the organ and it was quickly frozen in liquid nitrogen. The frozen organ was then placed in a zip bag, frozen in liquid nitrogen and then stored at −80° C.

### Study of Inflammation Parameters

The study was carried out based on the behavior of the animals treated. Specifically, inflammation causes exacerbated agitation in animals.

### Results

The results on the observation of the symptoms of inflammation are presented in tables 8 to 10 (before injection of composition C1) and in tables 11 to 15 (after injection of composition C1). In these tables:
the percent change in weight is calculated as follows: (DX×D0)/D0)*100)
Stool consistency is evaluated as follows: 0=normal; 2=soft; 3=diarrhea
Rectal bleeding is evaluated as follows: 0=none; 1=light; 2=moderate; 3=substantial; 4=profuse.

TABLE 8

| | | Days | | | | | |
|---|---|---|---|---|---|---|---|
| | | D3 | | | D4 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 10.7 | 0 | 0 | 12.14 | 0 | 0 |
| | Rat 2 | 11.1 | 0 | 0 | 13.02 | 0 | 0 |
| | Rat 3 | 9.5 | 0 | 0 | 16.07 | 0 | 0 |
| | Mean | 10.4 | 0 | 0 | 13.7 | 0 | 0 |
| DSS | Rat 4 | 5.2 | 0 | 0 | 8.52 | 0 | 0 |
| 2% | Rat 5 | 8.7 | 0 | 0 | 11.00 | 0 | 0 |
| | Rat 6 | 4.8 | 0 | 0 | 9.52 | 0 | 0 |
| | Mean | 6.2 | 0 | 0 | 9.7 | 0 | 0 |
| DSS | Rat 7 | 7.1 | 2 | 0 | 7.12 | 2 | 0 |
| 4% | Rat 8 | 3.6 | 2 | 0 | 5.57 | 2 | 0 |
| | Rat 9 | 6.2 | 2 | 0 | 6.49 | 2 | 0 |
| | Mean | 5.6 | 2 | 0 | 6.4 | 2 | 0 |

TABLE 9

| | | Days | | | | | |
|---|---|---|---|---|---|---|---|
| | | D5 | | | D6 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 11.7 | 0 | 0 | 14.04 | 0 | 0 |
| | Rat 2 | 13.65 | 0 | 0 | 16.51 | 0 | 0 |
| | Rat 3 | 14.75 | 0 | 0 | 17.74 | 0 | 0 |
| | Mean | 13.2 | 0 | 0 | 16.1 | 0 | 0 |
| DSS | Rat 4 | 8.52 | 0 | 0 | 10.03 | 0 | 0 |
| 2% | Rat 5 | 10.33 | 0 | 0 | 15.07 | 0 | 0 |
| | Rat 6 | 7.94 | 0 | 0 | 10.54 | 0 | 0 |
| | Mean | 8.9 | 0 | 0 | 11.9 | 0 | 0 |
| DSS | Rat 7 | 6.44 | 2 | 0 | 8.14 | 2 | 0 |
| 4% | Rat 8 | 8.20 | 2 | 0 | 9.64 | 2 | 0 |
| | Rat 9 | 5.84 | 2 | 0 | 7.40 | 2 | 0 |
| | Mean | 6.8 | 2 | 0 | 8.4 | 2 | 0 |

TABLE 10

| | | Days | | |
|---|---|---|---|---|
| | | D 7 | | |
| | | Weight | Stool | |
| | | change | consistency | Bleeding |
| | | (%) | (0/2/4) | (0-4) |
| Control | Rat 1 | 7.25 | 0 | 0 |
| | Rat 2 | 9.46 | 0 | 0 |
| | Rat 3 | 9.31 | 0 | 0 |
| | Mean | 8.67 | 0 | 0 |
| DSS 2% | Rat 4 | 4.59 | 2 | 0 |
| | Rat 5 | 6.50 | 2 | 0 |
| | Rat 6 | 4.06 | 2 | 0 |
| | Mean | 5.05 | 2 | 0 |
| DSS 4% | Rat 7 | 5.08 | 2 | 0 |
| | Rat 8 | 4.62 | 2 | 0 |
| | Rat 9 | 3.90 | 2 | 2 |
| | Mean | 4.53 | 2 | 1 |

TABLE 11

| | | Days | | | | | |
|---|---|---|---|---|---|---|---|
| | | D8 | | | D9 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 9.75 | 0 | 0 | 10.64 | 0 | 0 |
| | Mean | 6.53 | 2 | 0 | 3.13 | 2 | 0 |
| DSS | Rat 5 | 4.22 | 2 | 0 | 2.48 | 2 | 0 |
| 2% | Rat 6 | 5.38 | 2 | 0 | 2.80 | 2 | 0 |
| | Mean | 6.92 | 4 | 0 | 3.67 | 4 | 0 |
| DSS | Rat 8 | 1.36 | 4 | 2 | −1.10 | 4 | 4 |
| 4% | Rat 9 | 4.14 | 4 | 1 | 1.28 | 4 | 4 |
| | Mean | 9.75 | 0 | 0 | 10.64 | 0 | 0 |

TABLE 12

| | | Days | | | | | |
|---|---|---|---|---|---|---|---|
| | | D10 | | | D11 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 18.64 | 0 | 0 | 20.11 | 0 | 0 |
| | Mean | 3.17 | 2 | 0 | 5.87 | 2 | 0 |
| DSS | Rat 5 | 5.21 | 2 | 0 | 3.27 | 2 | 0 |
| 2% | Rat 6 | 4.19 | 2 | 0 | 4.57 | 2 | 0 |
| | Mean | 4.00 | 4 | 0 | 4.43 | 4 | 0 |
| DSS | Rat 8 | −0.88 | 4 | 4 | −0.36 | 4 | 0 |
| 4% | Rat 9 | 1.56 | 4 | 2 | 2.03 | 4 | 0 |
| | Mean | 18.64 | 0 | 0 | 20.11 | 0 | 0 |

TABLE 13

| | | Days | | | | | |
|---|---|---|---|---|---|---|---|
| | | D12 | | | D13 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 17.50 | 0 | 0 | 20.18 | 0 | 0 |
| | Mean | 3.73 | 2 | 0 | 3.63 | 2 | 0 |
| DSS | Rat 5 | 0.00 | 2 | 0 | −2.35 | 2 | 0 |
| 2% | Rat 6 | 1.87 | 2 | 0 | 0.64 | 2 | 0 |
| | Mean | 4.26 | 4 | 0 | 3.44 | 4 | 0 |
| DSS | Rat 8 | −1.30 | 4 | 0 | −2.18 | 4 | 0 |
| 4% | Rat 9 | 1.48 | 4 | 0 | 0.63 | 4 | 0 |
| | Mean | 17.50 | 0 | 0 | 20.18 | 0 | 0 |

TABLE 14

| | | Days | | | | | |
|---|---|---|---|---|---|---|---|
| | | D14 | | | D15 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 23.79 | 0 | 0 | 22.89 | 0 | 0 |
| | Mean | 6.53 | 0 | 0 | 2.87 | 0 | 0 |
| DSS | Rat 5 | 4.22 | 0 | 0 | −2.41 | 0 | 0 |
| 2% | Rat 6 | 5.38 | 0 | 0 | 0.23 | 0 | 0 |
| | Mean | 6.92 | 4 | 0 | 2.66 | 4 | 2 |
| DSS | Rat 8 | 1.36 | 4 | 2 | −2.31 | 4 | 2 |
| 4% | Rat 9 | 4.14 | 4 | 1 | 0.18 | 4 | 2 |
| | Mean | 23.79 | 0 | 0 | 22.89 | 0 | 0 |

TABLE 15

| | | Days | | | | | |
|---|---|---|---|---|---|---|---|
| | | D12 | | | D13 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 24.57 | 0 | 0 | 19.64 | 0 | 0 |
| | Mean | 3.20 | 0 | 0 | 0.93 | 0 | 0 |
| DSS | Rat 5 | −3.02 | 0 | 0 | −5.02 | 0 | 0 |
| 2% | Rat 6 | 0.09 | 0 | 0 | −2.04 | 0 | 0 |
| | Mean | 2.46 | 2 | 2 | −0.75 | 2 | 2 |
| DSS | Rat 8 | −3.31 | 2 | 2 | −2.95 | 2 | 2 |
| 4% | Rat 9 | −0.43 | 2 | 2 | −1.85 | 2 | 2 |
| | Mean | 24.57 | 0 | 0 | 19.64 | 0 | 0 |

It can be seen that the change in weight stabilized at D12 for the rats with DSS 2% and DSS 4%.

Regarding stool consistency, it becomes normal after 6 days of treatment with the composition according to the invention for the DSS 2% rats and after 8 days of treatment with the composition according to the invention for the DSS 4% rats.

Regarding rectal bleeding, it is absent in the DSS 2% rats, which means that there was recovery of intestinal function.

Regarding the analysis of the permeability of the intestinal membrane, the results of the in vivo tests 10 days after the injection of composition C1 into the DSS 2% rats, per FITC-dextran measurement, are presented in table 16.

TABLE 16

| | Control | DSS 2% | DSS 2% |
|---|---|---|---|
| | (FITC) - | FITC | FITC |
| | Rat 1 | Rat 5 | (Rat 6) |
| Plasma | 0.6519 | 0.6024 | 0.7431 |

It can be seen that the assay of FITC-dextran, 4 kDa in plasma makes it possible to clearly discern the impact of composition C1 according to the invention on rats that received 2% DSS, the fluorescence found in the blood being identical to that of the control.

Regarding the analysis of the permeability of the intestinal membrane, the results of the ex vivo tests 10 days after the injection of composition C1 into the DSS 2% rats, on isolated organs (permeability for isolated organs was carried out on a fragment of distal jejunum (5 cm) and on the ileum (5 cm), per FITC-dextran measurement, are presented in table 17.

TABLE 17

| Organs | | | | | |
|---|---|---|---|---|---|
| isolated | 30 min | 60 min | 90 min | 120 min | 150 min |
| Control | 34.4 | 103.6 | 254.6 | 526.9 | 861.5 |
| (FITC) - | | | | | |
| Rat 1 | | | | | |
| DSS 2% | 30.7 | 137.9 | 457.2 | 841.4 | 1101.0 |
| FITC | | | | | |
| (Rat 5) | | | | | |
| DSS 2% | 27.4 | 85.3 | 293.0 | 659.6 | 1030.0 |
| FITC | | | | | |
| (Rat 6) | | | | | |

It can be seen that for the rats treated with composition C1 according to the invention that received 2% DSS, the values are close to those observed in the control.

1. A liquid composition comprising:
at least one molecule covalently conjugated to a polylysine, and
at least one hydrophobic molecule which cannot be covalently conjugated to a polylysine, said molecule being encapsulated in a micelle formed by amphiphilic conjugates each consisting of at least one hydrophobic molecule covalently conjugated to a polylysine.

2. The composition as claimed in claim 1, characterized in that it also comprises:
at least one molecule conjugated to a polylysine by polymerization, said molecule and the polylysine forming a copolymer.

3. The composition as claimed in claim 1, characterized in that the one or more hydrophobic molecules which cannot be covalently conjugated to a polylysine are selected from among cholesterol, coenzyme Q10, farnesyl cysteine, curcumin, and mixtures thereof.

4. The composition as claimed in claim 1, characterized in that the one or more molecules covalently conjugated to a polylysine are selected from among fatty acids, antioxidants, vitamins and mixtures thereof.

5. The composition as claimed in claim 1, characterized in that the one or more molecules covalently conjugated to a polylysine are selected from among myristic acid, linoleic acid, palmitic acid, palmitoleic acid, lauric acid, oleic acid, orotic acid, spermine, taurine, pantothenic acid, azelaic acid, GABA, biotin, thioctic acid, ascorbic acid, acetic acid, propionic acid, butyric acid, pyruvic acid, lactic acid, alpha-tocopherol, pantothenic acid, glutathione and mixtures thereof.

6. The composition as claimed in claim 1, characterized in that the one or more micelles are formed at least by one or more alpha-tocopherol conjugates covalently conjugated to a polylysine and/or by one or more retinoic acid conjugates covalently conjugated to a polylysine.

7. The composition as claimed in claim 1, characterized in that the one or more micelles are formed at least by one or more fatty acid conjugates covalently conjugated to a polylysine.

8. The composition as claimed in claim 1, characterized in that it comprises at least one amino acid conjugated to a polylysine by polymerization, said amino acid and the polylysine forming a copolymer.

9. The composition as claimed in claim 8, characterized in that the amino acid conjugated to a polylysine by polymerization is selected from among cysteine and methionine.

10. The composition as claimed in claim 1, characterized in that the polylysine is poly-L-lysine.

11. The composition as claimed in claim 1, characterized in that the polylysine is linear and has a molecular weight of between 12,000 and 20,000 Da.

12. The composition as claimed in claim 1, characterized in that the polylysine has a bromide, chloride or TFA, trifluoroacetic acid, counterion.

13. A method for producing a composition as claimed in claim 1 comprising at least one hydrophobic molecule which cannot be covalently conjugated to a polylysine, encapsulated in a micelle formed by amphiphilic conjugates each consisting of at least one hydrophobic molecule covalently conjugated to a polylysine, characterized in that it comprises the following steps:
a. creating an amphiphilic premix: mixing conjugates of hydrophobic molecules covalently conjugated to a polylysine in an aqueous solution,
b. adding, to the amphiphilic premix, one or more hydrophobic one or more hydrophobic molecules which cannot be conjugated to a polylysine, and stirring so as to form a micelle of hydrophobic molecules covalently conjugated to a polylysine encapsulating the one or more hydrophobic molecules.

14. The production method as claimed in claim 13, characterized in that the stirring in step b. is carried out for between 5 and 20 minutes at a stirring speed of between 50 and 800 revolutions per minute.

15. The production method as claimed in claim 13, characterized in that it also comprises a step d. of separating the soluble and insoluble phases, in order to recover the soluble phase.

16. The production method as claimed in claim 13, characterized in that the premix of step a. also comprises one or more copolymers of a molecule conjugated to a polylysine by polymerization and/or, in a step c. which is carried out after step b. or after step d., one or more copolymers of a molecule conjugated to a polylysine are added to the mixture.

17. The production method as claimed in claim 13, characterized in that the premix of step a. also comprises one or more other molecules covalently conjugated to a polylysine and/or, after step b. or c., one or more other molecules covalently conjugated to a polylysine are added to the mixture.

18. The production method as claimed in claim 13, characterized in that it comprises a step of slow freeze-drying, spray-drying or dehydration of the liquid composition obtained previously in order to obtain a solid composition, followed by a step of solubilization of this solid composition in an aqueous solution.

19. A solid composition, characterized in that it is obtained by slow freeze-drying, spray-drying or dehydration of a composition as claimed in claim 1.
